(19) United States
(12) Patent Application Publication
(10) Pub. No.: US 2004/0193113 A1
(43) Pub. Date: Sep. 30, 2004

(54) EXPANDABLE BORE INJECTION NEEDLE

(75) Inventors: Edward M. Gillis, Santa Fe, CA (US); James Filice, Los Gatos, CA (US); Jim Brown, Los Gatos, CA (US); Andrew Ivan Poutiatine, Redwood City, CA (US); Charles Rampersaud, San Francisco, CA (US); Danny Argonza, San Jose, CA (US)

Correspondence Address: FACTOR & LAKE, LTD, 1327 W. WASHINGTON BLVD., SUITE 5G/H, CHICAGO, IL, 60607, US

(73) Assignee: DURECT CORPORATION, Cupertino, CA (US)

(21) Appl. No.: 10/707,983

(22) Filed: Jan. 29, 2004

Related U.S. Application Data
(63) Continuation of application No. 60/443,487, filed on Jan. 29, 2003.

(51) Int. Cl. A61M 5/178

(52) U.S. Cl. 604/164.01

(57) ABSTRACT

The present invention is a trocar/cannula assembly that is capable of delivering large-diameter objects/fluids into a body cavity interior, while minimizing the puncture footprint left after insertion. Generally, the invention could be a composite needle having a composite wall forming a trocar shaft with at least two rigid elements, and flexible material therebetween, which together at least partially define a trocar bore having a diameter, and a puncture tip. Together these elements enable the composite wall to flex outward so as to increase the diameter of the trocar bore. Additionally, the present invention could also be described as an expandable bore trocar/cannula that is made up of an expandable cannula body having an interior channel that is capable of flexing radially outward, and a trocar tip associated with the cannula body to facilitate the insertion of the trocar/cannula into a patient. Once the trocar/cannula is inserted, it may be flexed outward, and a medical implant may be inserted into the interior channel of the cannula body, and, in turn, into a body cavity of the patient.

### BACKGROUND OF INVENTION

1. Field of the Invention

The present invention is drawn generally to surgical devices, and, more specifically, to improved surgical devices for implanting or delivering a substance or object into the body of a patient.

2. Background Art

In the medical field there are numerous instruments specifically designed to penetrate bodily tissue so as to provide access to vessels, internal body cavities or organs. More specifically, an initial and primary activity during the performance of surgery is the creation of an access opening into the body at a predetermined surgical site.

In the past, such access openings were formed by the creation of a substantially large incision through the body wall or outer tissue, wherein the size of the incision would depend on the type of surgery, and accordingly, the surgical instruments involved. On completion of the surgical procedure, the large incision would be closed using conventional techniques. However, due to the traumatic nature of such open surgical procedures, the period of time required of the patient to completely heal was significant. In addition, the pain or discomfort during such recuperative period was a serious problem. Additionally, the psychological trauma and fearful anticipation caused by such surgery is, to some degree, proportional to the size of the incision made. The larger the incision, the greater the patient's fear of that incision, and thus the less chance the patient will be accepting of such a procedure.

Because of the above noted disadvantages, attempts have been made which were specifically directed towards new surgical procedures as well as instruments utilized in the support of such procedures. Currently, a popular alternative to open surgery is known as laparoscopic and/or endoscopic surgery, wherein a number of small openings, utilizing appropriate penetrating instruments, are formed to provide access into intended body cavities. Unlike the large incision required during open surgery, the much smaller access openings facilitate healing following the surgical and, as expected, result in significantly less discomfort to the patient. This is sometimes referred to as “keyhole” surgery.

To facilitate implantation of a substance or object, any number of instruments may be utilized to create the small openings into the body. One type of instrument is a trocar/cannula, which comprises a trocar or puncturing tip, and a cannula or tube. The trocar is placed into contact with an area of the skin to which access is desired and pushed through the skin until it has been breached, and the tip is present inside the body. Thereafter, the cannula acts like a conduit, offering access to the underlying tissue/cavity for the cannula, medical instruments, fluids, implanted devices and the like.

Typical trocar/cannula devices may be used in a variety of different ways to implement surgical techniques, including providing vascular access, or site-specific access to particular body localities. In use, generally, the trocar/cannula is inserted into the body at a predetermined position, and then, depending upon the nature of the trocar/cannula device, and upon the intended surgical operation, the trocar/cannula may be left in position as a conduit, or may be used to facilitate the insertion of a surgical implant. Alternatively, the trocar/cannula is put into the intended position and is expanded to facilitate the insertion of a surgical implant. The teachings of the present invention can be applied to this later expandable diameter trocar/cannula use.

Typical trocar/cannula devices, however, have significant drawbacks. In order to create sufficient clearance for some applications, such as, for example, instrument insertion or viscous fluid delivery, a significantly large puncture wound to allow access. In order to create a puncture of sufficient size, conventional trocars and cannulas had diameters of increasing sizes. With these larger diameters come a number of drawbacks, including reduced patient acceptance, longer healing times, and surgically related dangers such as bleeding and infection.

It is therefore an object of the present invention to provide a surgical device that has improved patient acceptance relative to its insertion.

It is a further object of this invention to provide a surgical device that reduces unwanted side effects from its insertion, including reducing wound size, healing times, and trauma related dangers such as infection.

These and other objects will become apparent to one of ordinary skill in the art in light of the specification, drawings, and claims appended hereto.

### SUMMARY OF INVENTION

The present invention comprises several embodiments of an expandable bore trocar. The expandable bore trocar can comprise a fluid-delivery conduit, making the trocar an expandable needle, or merely act as a trocar/cannula combination for facilitating the insertion of a substance or medical implant. In either case, the present invention diminishes the size of the puncture wound of a patient when using a trocar, while still allowing large-diameter fluid flow, such as viscous fluid flow, and large object or instrument insertion. After removal of the present invention from a patient, the size of the remnant wound, as well as the healing time required for that wound, are both diminished relative to prior art devices.

A composite needle structure according to the following description has a composite wall forming a trocar shaft, which has at least two rigid elements and a flexible material therebetween. The composite wall helps to at least partially define a trocar bore having a diameter, and a puncture tip. During operation, the composite wall is capable of flexing outward so as to increase the diameter of the trocar bore, and thus to increase the diameter of objects/fluid that can be inserted therein, while decreasing the impact of the puncture wound from the device after removal.

Another preferred embodiment of the present invention is an expandable bore trocar/cannula that includes an expandable cannula body having an interior, a trocar tip associated with the cannula body to facilitate the insertion thereof into a patient, and a medical implant for insertion into the interior of the expandable cannula body. To facilitate the insertion of the medical implant, the cannula body should be capable of flexing radially outward so as to increase or decrease the diameter of the cannula body. The cannula body could take on a number of different shapes, including an expandable spring, an expandable ribbon spring, a rolled cannula, and a rotating cannula.

### BRIEF DESCRIPTION OF DRAWINGS

FIG. 1 comprises a side view of an expandable needle according to the present invention;

FIG. 1A comprises a cross sectional view of the expandable needle at Plane A;

FIG. 1B comprises a cross sectional view of the expandable needle at Plane B;

FIG. 1C comprises a cross sectional view of the expandable needle at Plane C;

FIG. 2 comprises a side view of a split-splined expandable trocar/cannula according to the present invention;

FIG. 2A comprises a cross sectional view of the split-splined trocar cannula at Plane A;

FIG. 2B comprises a cross sectional view of the split-splined trocar cannula at Plane B;

FIG. 2C comprises a cross sectional view of the split-splined trocar cannula at Plane C;

FIG. 3 comprises a perspective view of a deliver apparatus that includes a split-splined cannula;

FIG. 4 comprises a longitudinal cross sectional view of a spring cannula;

FIG. 5 comprises a longitudinal cross sectional view of a ribbon spring cannula;

FIG. 6 comprises a lateral cross sectional view of a rolled cannula; and

FIGS. 7A-7D comprise different side operational views of a rotating cannula.

### DETAILED DESCRIPTION

While this invention is susceptible of embodiment in many different forms, there is shown in the drawings and will be described in detail, specific embodiments with the understanding that the present disclosure is to be considered as an exemplification of the principles of the invention and is not intended to limit the invention to the embodiments illustrated.

The following specification and claims utilize the terminology “surgical implant” and “medical implant” to describe a variety of devices that are inserted into a body cavity through a cannula. Such devices could include medical instruments, surgical tubes, fluid conduits, and the like. In view of the need for brevity, consistency, and clarity, however, the following specification and claims will maintain the use of the terms “surgical implant” and/or “medical implant” throughout, while simultaneously incorporating all of the above possibilities, as well as their equivalents.

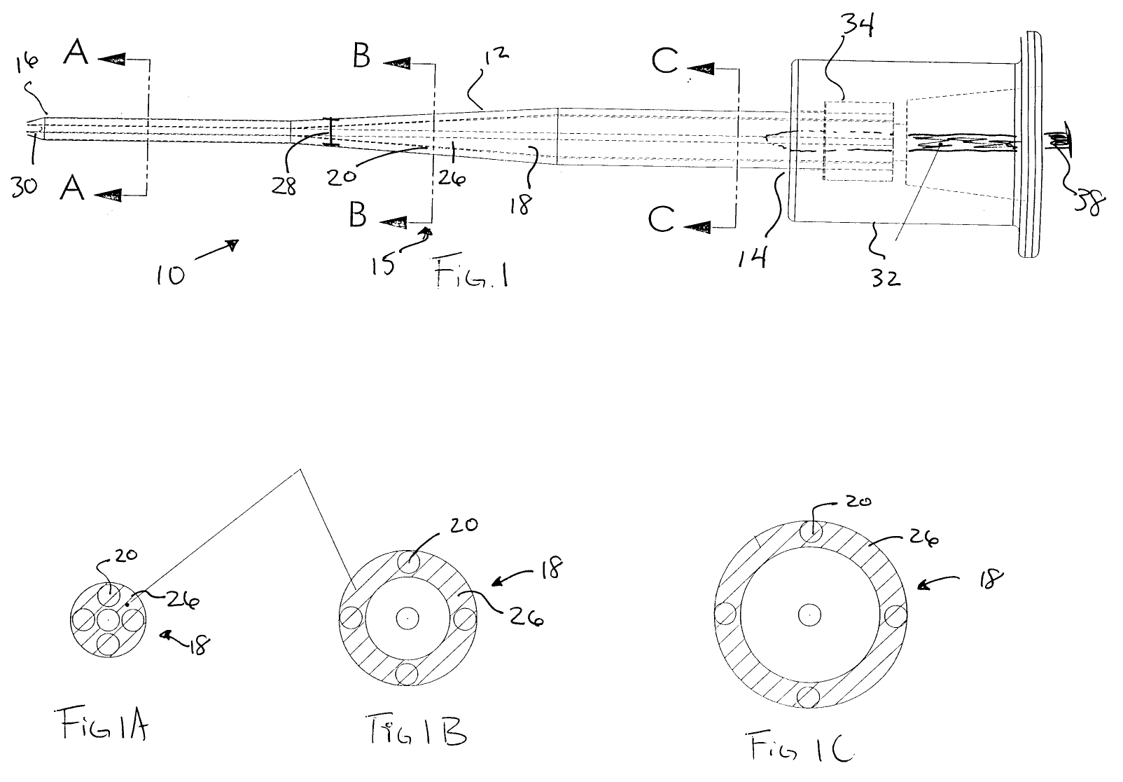

In one preferred embodiment, the teachings of the present invention can be incorporated into a trocar/cannula that is used as a conduit for surgical operations. In this embodiment, shown in FIG. 1, liquid delivery expandable trocar 10 comprises composite needle 12 having proximal end 14 fluidically coupled to fitting 32 at fitting junction 34, so as to allow fluids/instruments to be inserted into composite needle 12 through insertion recess 36 in fitting 32. Composite needle 12 comprises composite wall 18 having splines 20 running the length of composite needle 12, from proximal end 14 to distal end 16, and flex material 26 connecting splines 20 together, giving a substantially cylindrical, but tapered shape to composite needle 12. To form the tapered shape, composite needle 12 additionally comprises interior diameter 28, which narrows from proximal end 14, toward distal end 16, culminating at puncture tip 30.

Splines 20 of composite wall generally comprise long, wire-like cylinders having shaft 22 portions, and tapered ends 24. Splines 20 are preferably constructed from rigid materials, such as plastics, ceramics, metals, and composites thereof. These materials give splines 20 a rigid or semi-rigid structure so that splines 20 exhibit sufficient longitudinal strength (from distal end 16 to proximal end 14 of needle 12) to withstand the force required to puncture the tissue of a patient. In order to further facilitate such an operation, tapered ends 24 of splines 20 (located at distal end 16 of needle 12) join together into a single peak to help form puncture tip 30.

Splines 20 are joined together using flex material 26. Splines 20 may be connected by individual sheets of flex material 26, forming a repeated wall structure of splines 20 and then flex material 26, repeated around the circumference of composite wall 18. Alternatively, splines 20 could be incorporated into a single, tapered cylinder of flex material 26, with each spline 20 being at least partially embedded into flex material 26. Either way, splines 20 and flex material 26 together form composite wall 18 of needle 12. Flex material 26 is capable of resiliently expanding the distance between splines 20, while maintaining the structural integrity of composite wall 18. In order to do so, flex material 26 should be constructed from a flexible, resilient, and biocompatible material, such as a natural or synthetic elastic material such as silicon, rubber, elastomeric neoprene, latex, elastomeric plastics etc.

Composite needle 12 is shown in cross-section in FIGS. 1A, 1B, and 1C, wherein the specific cross sections correspond to the planes marked as A, B, and C, respectively, in FIG. 1. Plane A is located near distal end 16, Plane B near central portion 15 of the needle 12, and Plane C near proximal end 14. As can be seen in those cross sectional portions, splines 20 are spaced intermittently within composite wall 18. In a preferred embodiment, composite needle 12 includes four or more splines 20 to provide longitudinal rigidity to composite needle 12. Depending upon the size and configuration of splines 20, as few as one or as many as hundreds of splines 20 may be incorporated into composite wall 18, as long as sufficient rigidity is imparted to composite needle 12 for insertion of needle 12 into a patient.

In its relaxed position, and as shown in progression from FIGS. 1A to 1C, interior diameter 28 of composite needle 12 increases from distal end 16 to proximal end 14. By having an interior diameter 28 as small as possible at distal end 16 of composite needle 12, the puncture wound caused by and general profile of the composite needle 12 is smaller than conventional, high-capacity needle bores, which, in turn, increases patient acceptance of needle insertion, and decreasing the damage caused thereby.

The composite needle 12 structure is coupled with fitting 32 at fitting junction 34 to create a unitary structure. Fitting 32 provides a gripping structure for facilitating the insertion of expandable trocar 10 into a patient, as well as providing an access point for coupling external devices such as a syringe to expandable trocar 10. Fitting 32 comprises any number of known conventional fitting structures, such as the Luer ®Fitting.

Once assembled, expandable trocar 10 may be used in a variety of surgical techniques. In operation, a desired location for insertion of trocar 10 is selected, and an operator, handling trocar 10 by fitting 32, places puncture tip 30 into contact with the treatment area, and, by applying pressure, inserts trocar 10 into the selected location. The longitudinal strength provided by splines 20 to composite needle 12, as well as the sharp point provided by tapered ends 24, allow for easy insertion of trocar 10, with a minimal puncture footprint. Once inserted, an external device, such as a syringe, can be coupled to insertion recess 36 of fitting 32, and used to inject a fluid, for example, into composite needle 12. Depending upon several factors, including the viscosity of the injected fluid, the volumetric flow rate of injection, composite wall 18 of composite needle 12 can flex outward, increasing interior diameter 28 so as to accommodate the incoming fluid for delivery. The amount of flex exhibited by composite wall 18 will be proportional to the force applied to delivery the fluid. As composite wall 18 flexes outward and increases interior diameter 28, the puncture footprint created in the patient's tissue also increases.

After delivery of the fluid is completed, composite wall 18 will retract back to its original shape, and trocar 10 can be removed. As skin, by its nature, is resilient and elastic, the size of the puncture wound will retract back to its original, smaller footprint, easing patient recovery and minimizing damage to the tissue.

One related benefit to the expandable nature of the composite wall 18 of expandable trocar is in the delivery of extremely viscous fluids. As the viscosity of a fluid increases, the amount of force required to deliver that same fluid, through the same bore diameter, increases. In fact, for very viscous fluids, so much force may be required that delivery of the fluid by conventional hand means (i.e. fluid bulbs or syringes) may no longer be possible. In order to decrease the force required, therefore, the viscosity of the fluid must be increased, or the diameter of the injection bore increased. The present invention allows the delivery of a variety of fluids of differing viscosities, while still allowing for a small puncture footprint and the use of conventional, syringe-type delivery methods.

In some cases, it may be desirable to utilize splines 20 that do not, by themselves, have the longitudinal strength to facilitate insertion of trocar 10 into a patient. For example, in the delivery of extremely viscous fluids to a patient, a significant degree of flex may be required from composite wall 18 to accommodate the delivery, necessitating the use of less rigid splines 20. Alternatively, expandable trocar 10 could comprise a structure made entirely of flex material 26. In such cases, it is preferred to include stylet 38 (shown in FIG. 1) at the center of composite needle 12 to help facilitate insertion. Stylet 38 comprises a solid trocar device having a small puncture footprint. Typically, stylet 38 is formed from a rigid material, and has a puncture tip, a cylindrical shaft, and a flat, broad head to facilitate removal.

When expandable trocar 10 is utilized with stylet 38, the operation of the device is slightly altered. In such an embodiment, stylet 38 is placed into the center of composite needle 12, and expandable trocar 10 is inserted as described above. After insertion, stylet 38 is removed, leaving the flexible walls of composite wall 18 within the puncture footprint to facilitate the delivery of fluids and/or surgical or medical implant thereto. Once done with the particular procedure, expandable trocar 10 is removed.

The above-described device combines both the functions of creating an access point for the implanting physician, and providing a conduit for surgical implantation. In some surgical situations, however, it is preferred to utilize a medical implant for the surgical operations. Highly specialized implants, as well as cheaper implant alternatives to the expandable trocar 10 described above, can both provide beneficial surgical alternatives.

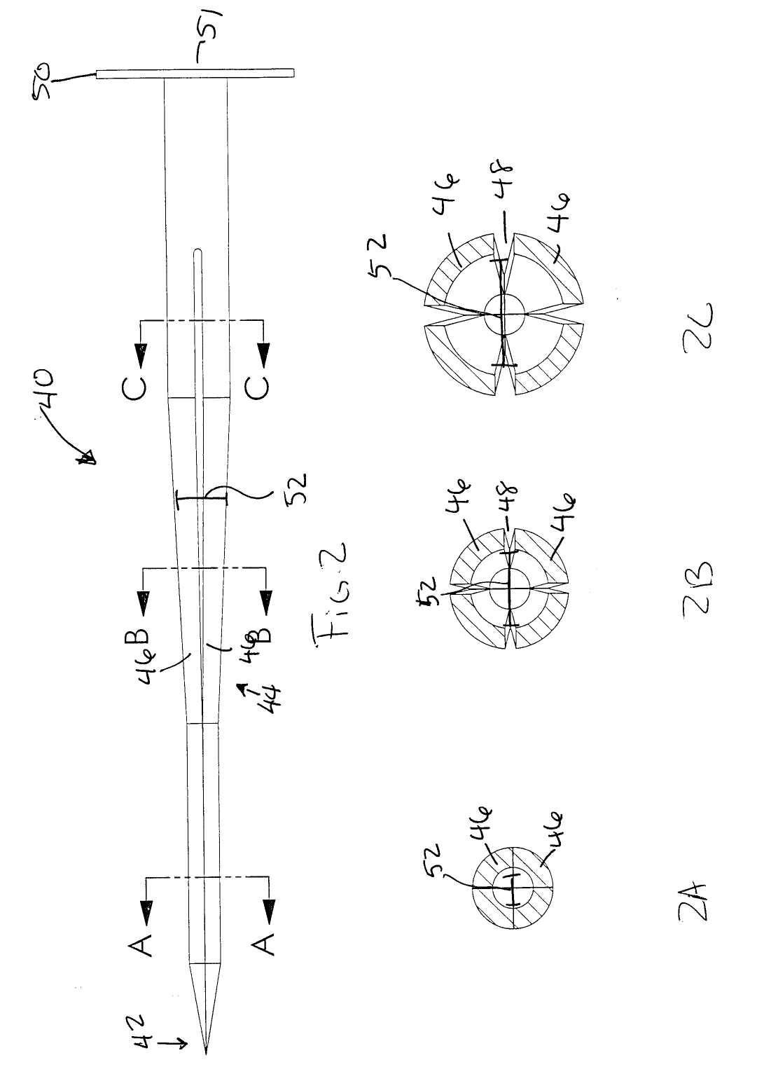

A second possible embodiment of the present invention is shown in FIG. 2. In this embodiment, an expandable metal cannula 40 may be utilized to create a small footprint puncture wound in a patient, and then to facilitate the insertion of a medical implant having a larger diameter into the wound for continued surgical use. Expandable metal cannula 40 is shown in one preferred embodiment in FIG. 2 as having puncture tip 42, shaft 44, and retraction head 50. Expandable metal cannula 40 with this design can be manufactured from a single, rigid material, if desired, although composite materials could operate similarly. For example, cannula 40 can be manufactured from stainless steel, stainless steel alloys, titanium, titanium alloys, or nickel-titanium alloys. Expandable metal cannula 40, as with expandable trocar 10 described above, has a tapered shape such that inside diameter 52 of cannula 40 decreases from retraction head 50 to puncture tip 42.

Shaft 44 of cannula 40 comprises split splines 46 with separation 48 between each spline 46. Preferably, shaft 44 has four splines 46, but could similarly operate with as little as two splines 46, and as many as hundreds. Separations 48 allow for splines 46 to be expanded outwards after insertion, increasing inside diameter 52 of cannula 40 from puncture tip 42 back as far as separation 48 extends. The material selected for cannula 40, of course, dictates the degree of expansion that is possible.

Expandable cannula 40 is shown in cross section in FIGS. 2A, 2B and 2C, which correspond to the cross-sectional planes designated in FIG. 2 as A, B and C, respectively. As can be seen, split splines 46 are preferably spaced evenly around the circumference of expanded cannula 40. The even spacing ensures that the force exerted upon surrounding tissues upon outward expansion of split splines 46 is approximately equal in all directions. Although such a configuration is preferred, it is possible to have a similarly effective device without the equal force distribution. For example, split splines 46 could each have a differing arc length such that one side or one spline was larger than the other remaining splines. Alternatively, only certain splines could be flexible, with, for example, a pair of opposing splines being flexible, with the alternating pair of splines being inflexible. In any case, and as with the expandable trocar, inside diameter 52 increases in size from puncture tip 42 to retraction head 50 when expandable cannula 40 is in a relaxed position.

Retraction head 50 comprises a substantially circular rigid piece of material, preferably made of the same material as shaft 44. Retraction head 50 additionally includes bore 51 (not shown) there through to enable the insertion of medical implant 54 into the interior portion of the cannula 40.

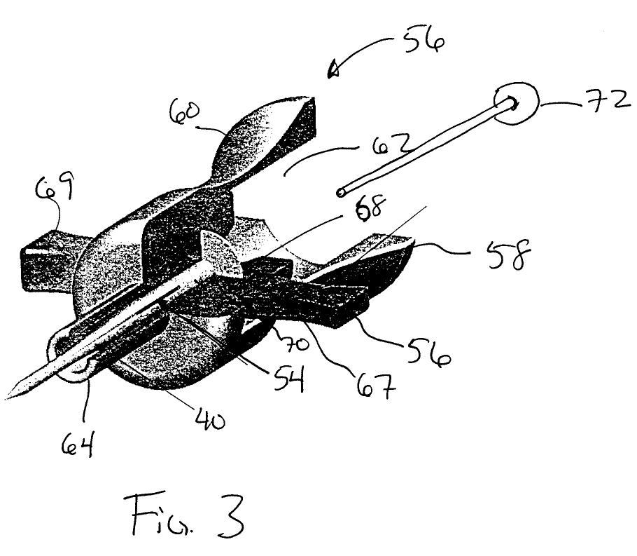

In operation, expandable cannula 40 can be utilized to effectively deliver medical implant 54 (shown in FIGS. 3C-3F). In order to do so, expandable cannula 40 is preferably associated with delivery apparatus 56 for facilitating the insertion of both expandable cannula 40, as well as medical implant 54, and removal of cannula 40, all while maintaining an optical shield from the patient.

Delivery apparatus 56, shown in FIG. 3, includes delivery shell 58, which incorporates withdrawal mechanism 66 therein. Delivery shell 58 comprises body 60, having channel 62 therein for withdrawal mechanism 66 (discussed below), and nose 64. Body 60 comprises a substantially cylindrical structure that is open from the top, and comprises the main gripping and support portion of delivery apparatus 56. Body 60 is constructed, therefore, from rigid type materials such as plastics, composites, metals, and the like. Body 60 includes an aperture in its bottom portion, which is associated with nose 64.

Nose 64 comprises a narrower cylindrical structure than body 60, and preferably comprises a tunnel-like structure approximating a diameter just larger than expandable cannula 40. Nose 64 provides a guide for the directed insertion of expandable cannula 40, as well as providing a visual shield during the insertion. Additionally, as retraction head 50 of expandable cannula 40 is integrated into withdrawal mechanism 66, as will be explained further below, the narrowing of the interior diameter from body 60 to nose 64 provides a depth stop for the insertion distance of expandable cannula 40. Generally, nose 64 is constructed from the same or similar materials as body 60.

Withdrawal mechanism 66 comprises central portion 67 having head recess 68, and handles 69 extending from either side of central portion 67. Head recess 68 accommodates the insertion of retraction head 50 of expandable cannula 40 so that, during use, withdrawal mechanism 66 can facilitate the retraction of cannula 40 from the patient. Withdrawal mechanism 66 additionally includes center hole 70 for the insertion of medical implant 54, and eventually for insertion of plunger 72. Center hole 70 lines substantially up with bore 51 of retraction head 50 to enable insertion of medical implant 54 all the way through into expandable cannula 40.

Handles 69 of withdrawal mechanism 66 are aligned within channel 62 of base 60 to enable the vertical movement of withdrawal mechanism 66. Handles 69 therefore are constructed from a rigid or semi-rigid material so as to enable the easy manipulation of withdrawal mechanism 66.

In operation, delivery apparatus 56 is assembled by inserting expandable cannula 40 into nose 64 of delivery shell 58, and associating retraction head 50 with head recess 68 of withdrawal mechanism 66. Handles 69 of withdrawal mechanism 66 are pushed all the way down in channel 62, allowing puncture tip 42 of cannula 40 to extend to its maximum distance out of nose 64. With cannula 40 fully extended, delivery apparatus 56 is placed into contact with a desired area of application on a patient, and pressed down to insert cannula 40 into the patient's tissue.

Once cannula 40 is inserted its maximum distance, medical implant 54 is inserted into center hole of withdrawal mechanism, through bore and into the interior portion of expandable cannula. Implant 54 is pushed into and through bore using plunger 70, by providing downward force using plunger head 72. Generally, medical implant has a greater interior diameter than expandable cannula so that, after insertion, split splines 46 of cannula 40 expand outward to accommodate the increased diameter of medical implant 54. As split splines 46 expand outward, they in turn increase the diameter of the puncture through the patient's skin, stretching the skin outwards to accommodate the diameter of medical implant 54.

After insertion of medical implant 54 is complete, withdrawal mechanism 66 is pulled away from the patient's skin using handles 68, keeping nose 64 in contact with the tissue as a visual shield. Throughout the withdrawal process, plunger head 72 is kept in its inserted position to maintain medical implant 54 within the skin. As withdrawal mechanism 66 is pulled back, retraction head 50 of expandable cannula 40 is also pulled back, retracting cannula 40 from the patient's skin. Because split splines 46 are flexible, medical implant 54 is maintained in its inserted position while cannula 40 is retracted around implant 54 and away from the body. Thereafter, the entire delivery apparatus 56 can be removed, leaving the inserted implant 54 in place.

The above descriptions have been based on a single type of expandable cannula 40. There are, however, a number of different structures that could similarly be used to accomplish the same or similar results described above, that is a small puncture footprint that can be expanded to accommodate a larger-diameter medical implant. These alternative structures could comprise, for example, an expandable spring structure, a ribbon spring structure, a rolled cannula, or a rotating cannula embodiment. Each of these embodiments will be discussed below.

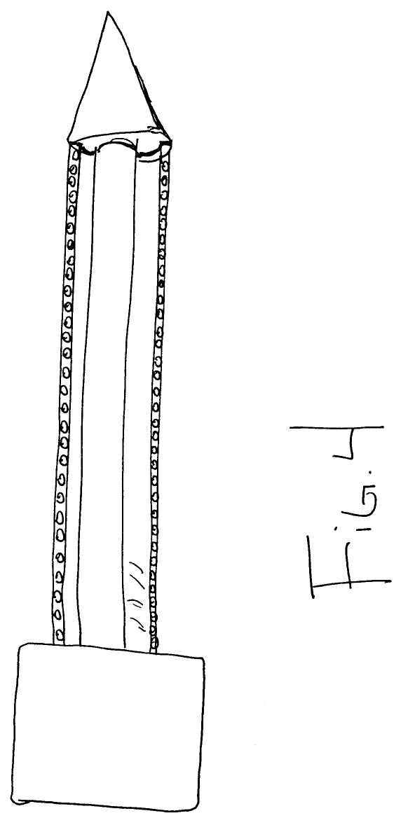

An expandable spring cannula embodiment is shown in longitudinal cross section in FIG. 4. Spring cannula 100 is shown as comprising spring wire 102 and cannula wall 104, surrounding trocar 110. Spring wire 102 and cannula wall 104 can comprise a composite wall structure, as discussed above relative to the expandable trocar/needle embodiment. Alternatively, spring wire 102 could be an over wire structure, associated only with the external portion of cannula wall 104, or an under wire structure, associated only with the internal portion of cannula wall 104. Spring wire 102 comprises a relatively stiff spring-like structure that winds circumferentially and spirally around with cannula wall 104. Cannula wall 104 comprises a resilient, flexible and biocompatible material. Together, these structures form the present spring cannula 100, which has a first end 106 and a second end 108.

In operation, generally, spring cannula 100 with trocar 110 is inserted into a patient. Once in place, first end 106 and second end 108 are rotated in opposite directions relative to one another, expanding spring wire 102, and, in turn, cannula wall. Once spring cannula 100 is expanded, trocar 110 can be removed, and any type of medical or surgical implant can be inserted.

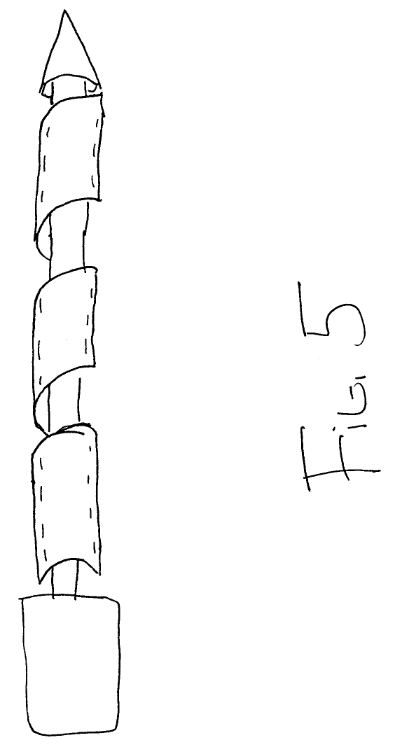

The ribbon-spring embodiment operates in a similar manner, and is shown in FIG. 5, again in longitudinal cross-section. Ribbon spring 120 is shown as comprising a simple strip of material wound having first end 122 and second end 124, wherein the ribbon spring 120 is circumferentially and spirally around trocar 126. Preferably, ribbon spring 120 is constructed from a rigid to semi-rigid material so as to provide longitudinal strength to the cannula. Further, ribbon spring 120 should be constructed from a biocompatible material so as to ensure safe and prolonged use is possible.

In operation, and as with the spring cannula 100 above, ribbon spring 120 and trocar 126 are associated together, and inserted into a patient. Once in operative position, ribbon spring 120 is rotated at first end 122 and second end 124 in opposite directions, expanding ribbon spring 120 outward, and, in turn, expanding the footprint of the puncture made into the skin of the patient. Once ribbon spring 120 is in an expanded position, trocar 126 can be removed and medical implant inserted, as needed.

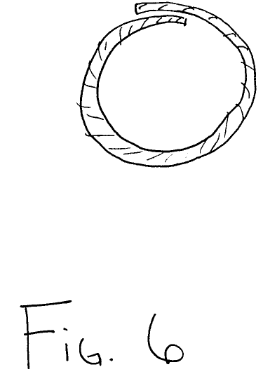

A related embodiment, the rolled cannula embodiment, is shown in FIG. 6. Rolled cannula 130 is shown in lateral cross section as having cannula wall 132. As can be seen, cannula wall 132 preferably comprises a single sheet of material that is wound into a cylinder, with a slight amount of overlap near the ends of the sheet of material, forming overlap area 134. Cannula wall 132 is formed from a rigid to semi-rigid material so as to provide longitudinal strength as well as providing a consistent and reliable cylindrical structure. Cannula wall 132 encloses central channel 136 into which a trocar can be inserted.

In operation, a trocar is associated with central channel 136 of cannula wall 132, and then rolled cannula 130 and the trocar can be inserted into a patient. Once in place, cannula wall 132 can be expanded outward via force or through another similar means to, in turn, expand the diameter of the puncture into the patient. As the diameter is expanded, the size of overlap area 134 is decreased. After cannula wall 132 has been expanded sufficiently far enough, the trocar is removed and a medical implant may be inserted.

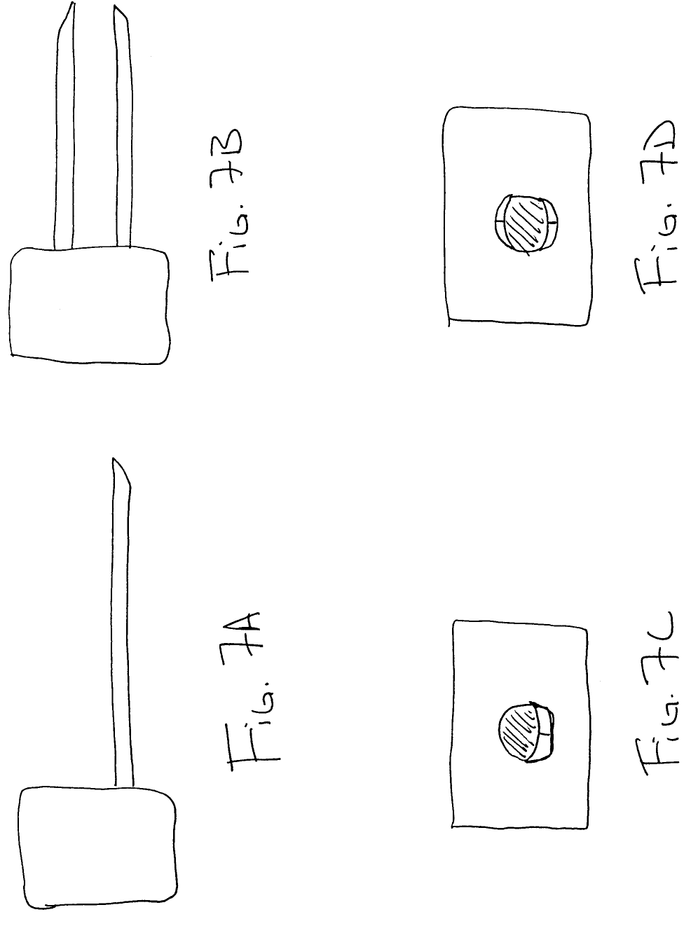

A final, alternative embodiment is shown in FIGS. 7A-7D as the rotating cannula embodiment 140. Rotating cannula embodiment 140 comprises a puncture cannula 142, and a rotating cannula 144, both of which include a shaft portion 146, and a trocar portion 148. As can be seen, shaft portion 146 comprises a substantially cylindrical tube for the insertion of items into the puncture incision, and trocar portion 148 comprises a longer, narrower section having a puncture tip 150. Trocar portion 148 preferably has an arc-like shape that mimics the curvature of shaft portion 146. Rotating cannula 144 is associated with puncture cannula 142 so that puncture cannula 144 can rotate in a circumferentially related path to either be aligned with puncture cannula 142 (shown in FIGS. 7A, 7C), or be rotated to a non-aligned position (shown in FIGS. 7B, 7D). Each of these elements is preferably constructed from a rigid or semi-rigid material.

In operation, rotating cannula embodiment 140 initially has puncture cannula 142 and rotating cannula 144 substantially aligned for insertion into a patient. Once inserted into a patient, rotating cannula 144 is rotated away from the aligned position into a position where the trocar portion 148 of rotating cannula 144 is in a diametrically opposed position to puncture cannula 142. As rotating cannula 144 is rotated, the diameter of the puncture wound in the patient increases, allowing for the insertion of a medical implant therein that has a larger diameter than the originally created puncture wound.

Any number of other embodiments may also accomplish the same functions as described above, as would be known by one of ordinary skill in the art.

The foregoing description merely explains and illustrates the invention and the invention is not limited thereto except insofar as the appended claims are so limited, as those skilled in the art that have the disclosure before them will be able to make modifications without departing from the scope of the invention.

1. An expandable bore trocar/cannula, comprising:
A composite needle forming a cannula shaft and a puncture tip; and
The composite needle comprising at least one rigid element, and flexible material connected to the at least one rigid element, wherein the rigid elements and the flexible material together form the cannula shaft, and the rigid elements form the puncture tip.

2. The device according to claim 1, wherein the rigid elements comprise a material selected from the group consisting of plastics, ceramics, metals, and combinations and composites thereof.

3. The device according to claim 1, wherein the flexible material comprises a material selected from the group consisting of natural or synthetic elastic material, silicon, rubber, elastomeric neoprene, latex, and elastomeric plastics.

4. The device according to claim 1, wherein the cannula shaft comprises a diameter, wherein the diameter is smaller at a proximal end proximate the puncture tip than at a distal end distal from the puncture tip.

5. The device according to claim 1, wherein the composite needle comprises four rigid elements.

6. The device according to claim 1, further comprising a fitting into which a distal end of composite needle is inserted, wherein the distal end is distal from the puncture tip, and the fitting includes an access bore that corresponds with a cannula bore formed by the cannula shaft.

7. The device according to claim 6, wherein the fitting comprises an insertion recess, wherein the insertion recess is configured to connect to fluid-insertion devices.

8. An expandable bore trocar/cannula, comprising:
An expandable cannula body, wherein the cannula body has an interior channel with a diameter, wherein the cannula body is capable of flexing so as to increase or decrease the diameter of the cannula body; and
Means for inserting the cannula body into a patient.

9. The device according to claim 8, wherein the cannula body comprises a composite needle having at least one rigid element, and flexible material connected to the at least one rigid element, wherein the rigid element and the flexible material together form the cannula body, and the inserting means comprise the rigid elements, which form a puncture tip.

10. The device according to claim 8, wherein inserting means comprises a stylet configured for insertion into and withdrawal from the interior channel of the cannula body.

11. An expandable bore trocar/cannula, comprising:
An expandable cannula body, wherein the cannula body has an interior channel with a diameter, wherein the cannula body is capable of flexing so as to increase or decrease the diameter of the cannula body; and
A stylet configured for insertion into, and withdrawal from the interior channel.

12. The device according to claim 11, wherein the cannula body comprises a composite needle having at least one rigid element, and flexible material connected to the at least one rigid element, wherein the rigid element and the flexible material together form the cannula body.

13. The device according to claim 11, wherein the cannula body comprises at least two rigid elements.

14. The device according to claim 13, wherein the cannula body comprises four rigid elements.

15. The device according to claim 11, wherein the cannula body comprises an expandable spring cannula, the spring cannula comprising at least one rigid element formed in the shape of a helix and having a center channel, and a flexible material attached to the rigid element to form at least a portion of a cylinder surrounding the center channel.

16. The device according to claim 11, wherein the cannula body comprises a rotating cannula, the rotating cannula having at least a first and a second rigid element which comprise a portion of a cylinder arc, the first and second rigid elements being concentric to a center line of the rotating cannula, with the second rigid element being capable of rotating around the center line.

17. The device according to claim 11, wherein the cannula body comprises a ribbon spring, the ribbon spring comprising at least one rigid element formed in the shape of a helix and having a center channel.

18. The device according to claim 11, wherein the cannula body comprises a rolled cannula, the rolled cannula comprising at least one sheet of rigid material having a first longitudinal side and a second longitudinal side, the sheet being formed into the shape of a cylinder and having a center channel with a diameter, the sheet overlapping at the first and second longitudinal sides so as to allow the diameter to be varied.

19. A medical insertion device for use with one of a trocar and a cannula, comprising:
A delivery apparatus having a delivery shell configured to be grasped by a user;
Wherein the delivery shell includes a channel, and a nose extending outward from the shell, the nose having a bore therethrough aligned with the channel, wherein the channel and bore are configured for insertion of the trocar or cannula therethrough; and
The delivery apparatus further comprising a withdrawal mechanism proximate the channel, which is configured to cooperate with a retraction head of the trocar or cannula, and which includes at least one handle, wherein the handle facilitates the passage of the trocar or cannula through the channel and bore, and out of the delivery apparatus through the nose.
